(19) United States
(12) Patent Application Publication
(10) Pub. No.: US 2017/0212192 A1
(43) Pub. Date: Jul. 27, 2017

(54) NEONATE'S IMMOBILIZING RESTRAINER

(71) Applicant: Aspect Imaging Ltd., Shoham (IL)

(72) Inventors: Uri RAPOPORT, Moshav Ben Shemen, IL (US); Thomas Rangi SUTTON, Milano (IT); James William LUTHER, Milano (IT); Andrea BESANA, Seveso (IT); Barbara TORNAGHI, Monza (IT); Shmuel AZULAY, Tel Aviv (IL); Irad LEISER, Ramat Gan (IL)

(21) Appl. No.: 15/481,631

(22) Filed: Apr. 7, 2017

Related U.S. Application Data
(63) Continuation-in-part of application No. 14/050,531, filed on Oct. 10, 2013.
(60) Provisional application No. 62/325,235, filed on Apr. 20, 2016.

(51) Int. Cl. G01R 33/341; A61G 11/00; A61F 5/37

(52) U.S. Cl. CPC G01R 33/341; A61F 5/3776; A61G 11/00; A61F 5/3707; A61B 2503/045

(57) ABSTRACT

A neonate's immobilizer, said immobilizer comprising a lower torso segment having dorsal side and ventral side, an upper torso segment having the same, and a median navel segment in between, having a lateral side; said three segments are interconnected to form a continuous restraining shell along said dorsal side; said navel segment has a lateral side, wherein said navel segment's lateral side, lower and upper segments form an access aperture around said navel when restraining said neonate, said aperture is provided in size and shape for visual inspection and physical access to said neonate's navel.

### CROSS REFERENCE TO RELATED APPLICATIONS

This application is a continuation-in-part of U.S. patent application Ser. No. 14/050,531, filed on Oct. 10, 2013, and this application claims the benefit of and priority to U.S. Provisional Patent Application No. 62/325,235, filed on Apr. 20, 2016, the entire contents of which are incorporated herein by reference in their entireties.

### FIELD OF THE INVENTION

The present invention generally relates to the field of neonates immobilizing by a restrainer comprising a navel's infusion aperture. The invention also pertains to methods of immobilizing neonates by his restrainer whilst neonate's navel is continuously accessible for both visual inspection and medical treatment.

### BACKGROUND OF THE INVENTION

Central catheter is a thin, flexible tube or catheter placed in a larger vein or artery to deliver medications or necessary fluids to your baby. Central catheters also include umbilical venous and umbilical artery catheters which may be inserted into the vein or artery of the umbilical cord (belly button). Newborns that need special care, especially those hospitalized in neonatal intensive care unit (NICU), are frequently coupled and intubated to life sustaining and monitoring lines at all times, including during their MRI scanning, where their immobilization is required.

U.S. Pat. No. 6,992,486 discloses a magnetic resonance imaging system and a life sustaining incubator system used for neonates. The system includes restraining mechanisms for supporting, and immobilizing the infant and during MRI examination. U.S. Pat. No. 6,860,272 further discloses a device for diagnostic procedures when using an MRI unit, CT scan unit or x-ray unit, which comprises a patient resting surface to which a patient's body parts are firmly, but comfortably secured. The device further includes anchoring means for immobilizing the patient by applying a controllable pressure upon the patient's skeleton, joints and spine. The above described systems, however, do not provide a complete solution for immobilizing newborn whilst providing micro-environment surrounding in a secured manner, during MRI scanning, namely providing the infant a continuous life sustaining and monitoring lines. Hence, prior art does not disclose neonate's restraining means where neonate's belly button is continuously accessible for both visual inspection and medical treatment.

Kitterman et al., in Catheterization of umbilical vessels in newborn infants (1970) Pediatric Clinics of North America 17(4)895-91, which is incorporated herein as a reference, concluded that the use of an indwelling catheter in an umbilical vessel can lead to improved care of sick infants; however, it may also lead to serious complications, such as thrombosis, embolism, vasospasm, vascular perforation, vascular damage from hypertonic solutions, hemorrhage, bowel perforation following exchange transfusion, infection, electrical hazards etc.

Regarding infection hemorrhage risks related with this catheterization an umbilical arterial catheter is a direct connection to the aorta, and thus there is a constant risk of major and even fatal hemorrhage (see Kitterman et al.). It is extremely important to notice immediately if it becomes disconnected. This requires close nursing supervision at all times. Ideally, each infant with an indwelling umbilical arterial catheter should have a nurse assigned to care for him alone. Because the infant may pull or kick the catheter or its connecting tubing, the infant's arms and legs must be restrained. If significant bleeding occurs, the infant's blood volume should be restored as soon as possible by transfusion. Regarding infection risks related with this catheterization, the umbilical dressing should be changed and an antibiotic ointment applied daily; catheters should be removed from umbilical vessels as soon as they are no longer essential for the infant's welfare. In addition, care should be taken not to contaminate the catheter connections or syringes used for withdrawing or injecting blood or other fluids through the catheter.

Another difficulty during MRI scanning of a neonate can be noise in the environment of the MRI. MRI's are typically extremely loud machines, and neonates can be put under additional stress when subject to the MRI noise.

Therefore, there is a long felt and unmet need to provide an MRI-proof neonates immobilizing arrangement whereby neonate is effectively restrained whilst his/her navel is continuously accessible for both visual inspection and medical treatment. It can also be desired to provide a noise reduced experience for a neonate during an MRI.

### SUMMARY OF THE INVENTION

It is thus an object of the present invention to provide a neonate's immobilizing arrangement whereby the neonate is effectively restrained whilst his/her navel is continuously accessible for both visual inspection and medical treatment.

It is another object of the present invention to provide neonate's immobilizer as defined above, wherein this immobilizer comprises a lower torso segment having dorsal side and ventral side, an upper torso segment having the same, and a median navel segment in between, having a lateral side. The three segments are interconnected to form a continuous restraining shell along said dorsal side. The navel segment has a lateral side, wherein the navel segment's lateral side, lower and upper segments form an access aperture around the navel when restraining the neonate, the aperture is provided in size and shape for visual inspection and physical access to the neonate's navel.

It is another object of the present invention to provide MRI-proof neonate's immobilizer as defined above, wherein the immobilizer is made of nonmagnetic materials.

It is another object of the present invention to provide MRI-proof neonate's immobilizer as defined above, wherein the immobilizer additionally comprising at least one neonate's head restraining segment, e.g., a single head restraining member interconnected to the aforementioned three segments.

It is another object of the present invention to provide MRI-proof neonate's immobilizer as defined above, wherein the immobilizer is provided with an access aperture which allows intubation of an infusion system via the neonate navel during medical care and analysis, e.g., along MRI scanning

It is another object of the present invention to provide a neonate's immobilizer as defined in any of the above, wherein the immobilizer additionally comprises a protective element configured to at least partially and reversibly cover said access aperture.

It is another object of the present invention to provide a neonate's immobilizer as defined in any of the above, wherein the immobilizer additionally comprises at least one radiofrequency (RF) coil aligned with the neonate's head.

It is another object of the present invention to provide a neonate's incubator comprising a neonate immobilizer as defined in of the above.

It is another object of the present invention to provide a method for restraining a neonate characterized by steps of providing a neonate's immobilizer with a lower torso segment having dorsal side and ventral side, an upper torso segment having the same, and a median navel segment in between, having a lateral side; interconnecting said three segments are form a continuous restraining shell along said dorsal side; providing said navel segment with a lateral side; forming, by means of said navel segment's lateral side, lower and upper segments an access aperture around said navel, thereby when restraining said neonate, providing said aperture's size and shape for visual inspection and physical access to said neonate's navel.

It is another object of the present invention to provide a method as defined above, the method additionally comprises a step of constructing the immobilizer by MRI-proof materials.

It is another object of the present invention to provide a method as defined above, the method additionally comprises a step of providing the immobilizer with a neonate's head restraining segment.

### BRIEF DESCRIPTION OF THE DRAWINGS

In order to understand the invention and to see how it may be implemented in practice, a few preferred embodiments will now be described, by way of non-limiting example only, with reference to be accompanying drawings, in which:

FIG. 1A illustrates a top and side view of an immobilizer device restraining an infant according to one embodiment of the invention, and FIG. 1B illustrates the same, wherein the infant's navel is catheterized via an immobilizer's dedicated aperture aperture by a central catheter e.g., to the umbilical venous;

FIGS. 2A and 2B illustrate a perspective views of an MRD immobilizer device according to various embodiments of the invention; and

FIG. 3 illustrates a perspective view of an MRI-compatible neonate's incubator according to one embodiment of the invention, where neonate is placed within an immobilizer-incubator integrated system.

FIG. 4 is an illustration of a neonate immobilizer including noise reduction components, according to illustrative embodiments of the invention.

FIG. 5a-5B are an illustrations of a head segment of a neonate immobilizer, according to illustrative embodiments of the inventions

### DETAILED DESCRIPTION OF THE PREFERRED EMBODIMENTS

The following description is provided so as to enable any person skilled in the art to make use of the invention and sets forth the best modes contemplated by the inventor of carrying out this invention. Various modifications, however, will remain apparent to those skilled in the art, since the generic principles of the present invention have been defined specifically to provide means and methods for neonates immobilizing by a restrainer comprising a navel's infusion aperture

The terms ‘magnetic resonance device’ and MRD interchangeably applies to any medical device and analysis device, such as a magnetic resonance imaging (MRI) device, nuclear magnetic resonance (NMR) spectroscope, electron spin resonance (ESR) spectroscope, nuclear quadruple resonance (NQR) computerized tomography (CT), ultrasound (US) and any combination thereof. As used herein, the term “neonate” refers to any of the following: neonate, baby, infant, premature baby, child, newborn, human patient, and other mammalians (e.g., laboratory animals).

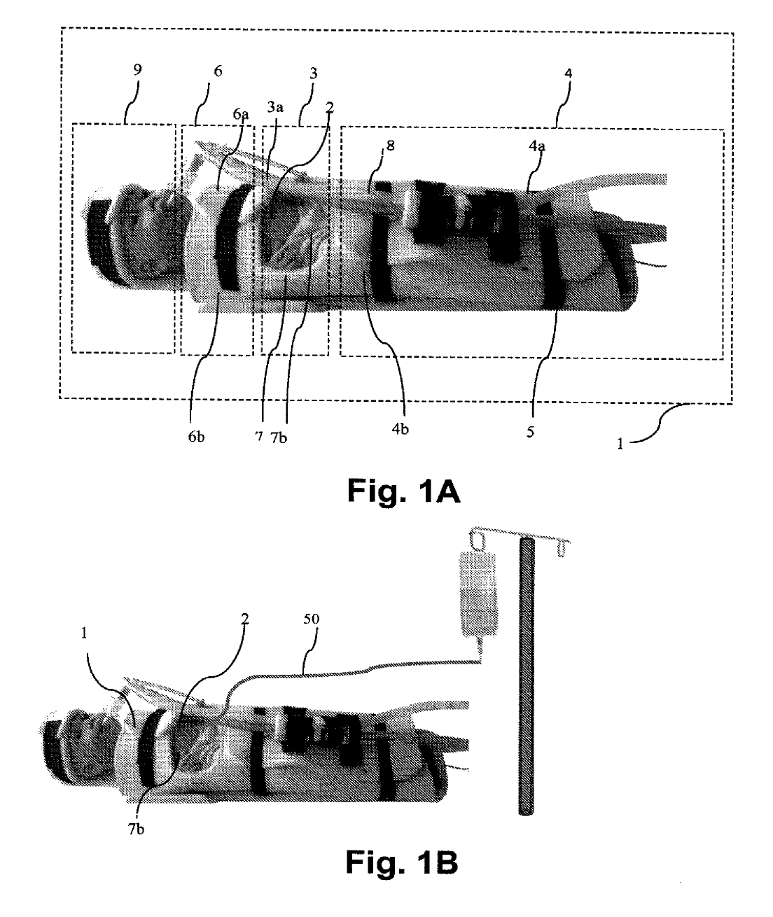

Reference is now made to FIG. 1A, which schematically illustrates a side and top view of an embodiment of the immobilizer (dashed block 1) which comprises a lower torso segment (dashed block 4) having dorsal side and ventral side, an upper torso segment (dashed block 6) having the same, and a median navel segment (dashed block 3) in between, having a lateral side. Those three segments are interconnected to form a continuous restraining shell along the dorsal side (4b, 6b). The navel segment has one or more lateral sides (7), wherein navel segment's lateral side (7), lower (4b) and upper (6b) segments form an access aperture (7b) around neonate's navel (2) when restrainer (1) is enveloping thereby immobilizing the neonate. Aperture (7b) is provided in size and shape which enables an effective, safe and convenient visual inspection and physical access to neonate's navel (2).

Reference is still made to FIG. 1A, illustrating an MRD-adapted immobilizer (1), here an MRI-proof restrainer (1) as a non-limiting example Immobilizer comprises segmented upper (left-) flanks (4a, 6a) and segmented lower (right-) flanks (4b, 6b) which are adapted to wrap the infant to a continuous shell. The immobilizing shall can be secured by beans of anchoring means (5), which are selected, e.g., from the group consisting inter alia one or more of the following: strap, strip, girth, belt, leash, lock, latch, fastener and any combination thereof.

Reference is now made to FIG. 1B, which schematically illustrates a side and top view of the immobilizer (1) as defined and described above. A central catheter (50), e.g., an infusion catheter, is introduced to infant' navel (2) via aperture (7b).

It is in the scope of the invention wherein the central catheter and/or infusion system is selected in a non-limiting manner from a group consisting, inter alia, infusion tube system, life support system, air and oxygen tubes, respirator tubes, intravenous tubes, ventilation tubes, catheter and any combination thereof. It is further in the scope of the invention wherein the access aperture (7b) allows coupling the central catheter and/or infusion system via neonate's navel for supplying the neonate element(s) selected in a non-limiting manner from a group consisting of fluids (e.g., blood, isotonic solutions), gasses (e.g., oxygen), medications and any combination thereof. It is further in the scope of the invention wherein access aperture (7b) allows connection to at least one physiological sensor to be connected or otherwise introduce to neonate's body.

It is further in the scope of the invention wherein immobilizer (1) is at least partially made from materials selected from the group consisting, inter alia, semi-flexible materials, semi rigid materials, rigid materials, flexible materials and any combination thereof.

In another embodiment of the present invention, immobilizer (1) comprises or otherwise provide in connection with at least one radiofrequency (RF) coil aligned with the neonate's head, bally or other predefined organ or location. Such a coil, according to yet another embodiment of eth invention, is integrated with immobilizer (1) or a segment thereof, appended to at least one portion of the immobilizer, provided in connection with immobilizer (1) or any combination thereof.

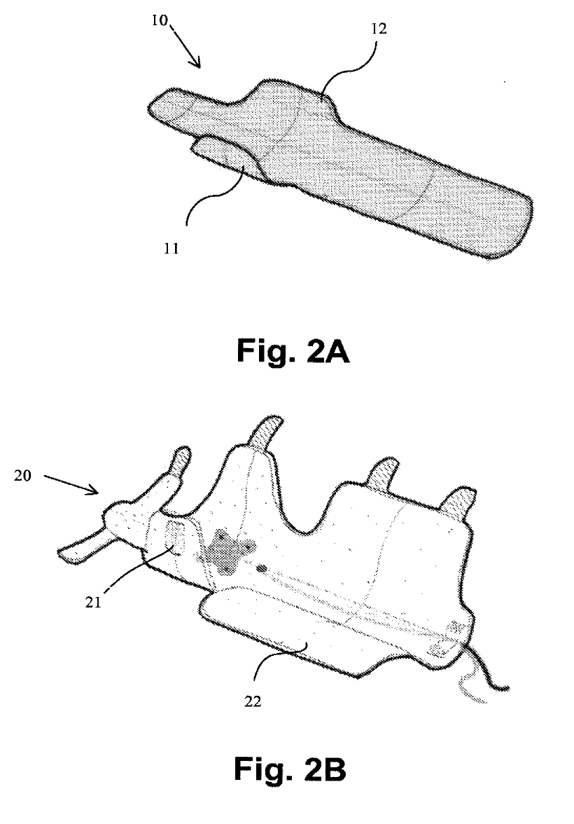

Reference is now made to FIG. 2A, presenting in an out-of-scale manner a perspective view of a rigid tray (10) for supporting immobilizer (1) according to yet another embodiment of the invention. Support tray (10) is configured to stabilize and support neonate torso, neonate's shoulders, supine, back and lower body. The support tray may further comprise semi flexible wings-like flanks (11, 12) for surrounding and raping neonate's body parts.

Reference is now made to FIG. 2B which illustrates a perspective view of an immobilizer (20) according to an embodiment of the present invention comprising semi flexible shell for enclosing and restraining a neonate in addition a semi rigid or fully rigid support tray (10, FIG. 2A) for further stabilizing the infant torso. Immobilizer 20 comprises semi flexible lateral wings-like flanks (21, 22) for safely rapping the neonate body, thereby forming an access aperture upon the neonate abdominal and more practically, upon the neonate navel.

In another embodiment, the shell and the support tray may be integrated as one unit for stabilizing and in parallel immobilizing the neonate body to the incubator.

The present invention further provides an MRD immobilizer device (e.g., immobilizer 1) adapted for immobilizing a neonate during MRI scan.

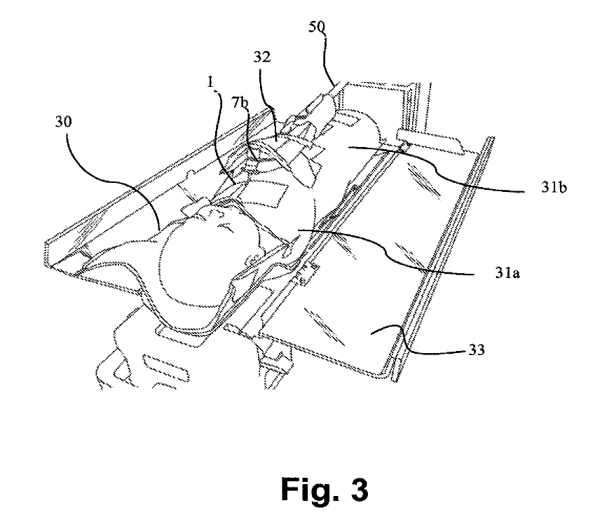

Reference is now made to FIG. 3 illustrates a perspective view of a neonate's incubator (30), herein its open configuration where canopy flank (33) is open, comprising or is in connection with a neonate immobilizer (1) as defined any of the above. The immobilizer comprising at least two segments (31a, 31b) defining a nave's aperture (7b), here cover with an aperture's cover (32). A central catheter (50) such as an infusion line is inserted under cover (32) via aperture (7b) to neonate's navel.

In another embodiment of the present invention, intravenous (IV) lines and monitoring cables are grouped at the feet end of the immobilizer. Vital signs (e.g., ECG and pulse oximetry) are monitored with this in vivo simultaneously medically treating and immobilizing system.

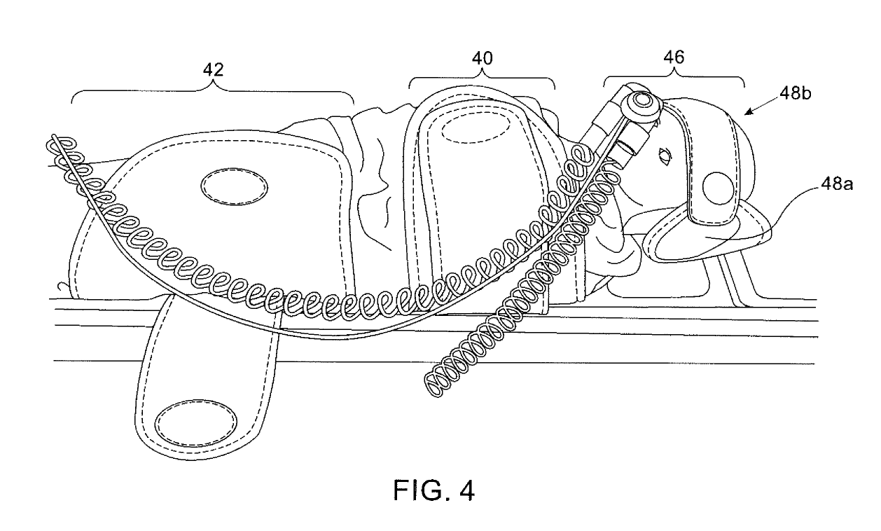

FIG. 4 is an illustration of a neonate immobilizer including noise reduction components, according to illustrative embodiments of the invention. FIG. 4 includes an immobilizer having an upper torso segment (40), a lower torso segment (42) and a head segment (46). The head segment (46) can include at least two noise reduction components (48a and 48b, generally 48). The noise reduction components 48 can be positioned such that when the head segment is closed around the neonate, the noise reduction components (48) cover the ears of the neonate. The noise reduction component (48) can be made of non-magnetic material. The noise reduction component (48) can be made of melamine and/or other noise reduction foam as is known in the art.

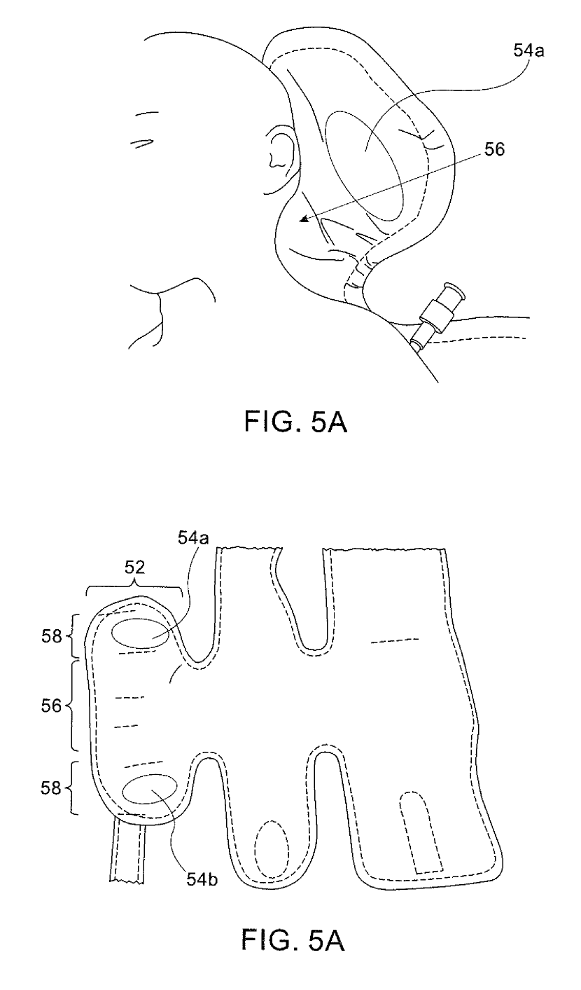

FIG. 5a-5B are an illustrations of a head segment (52) of a neonate immobilizer, according to illustrative embodiments of the inventions. The head segment (52) includes at least two noise reduction components (54a and 54b, generally 54). The head segment (52) can have a first portion (56) which the neonate's head rests on, and a second portion (58) that can be a flap that wraps around a forehead of the neonate and connects via a connector (e.g., Velcro, push pin, and other connectors as are known in the art to removable couple two fabrics together). When the flaps (58) wrap around the forehead of the neonate and connect it can cause the first portion (56) to bend such that the first portion (56) wraps around the ears of the neonate.

In some embodiments, the first portion (56) is made out of a material that allows the first portion to wrap around the ears of the neonate without the flaps (58) connected.

In various embodiments, the two noise reduction components (54) are circular shaped, shaped according to the ear of the neonate, square, rectangle, or any combination thereof. In some embodiments, the two noise reduction components (54) are removeable from the second portion (56). In some embodiments, the two noise reduction components (54) are sewn, glued, or permanently attached to the second portion (54). In some embodiments, the two noise reduction components are melamine (e.g., Basotec).

In some embodiments, the two noise reduction components (54) can reduce the noise by 2-8 db(A).

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

1. A neonate immobilizer for immobilizing a neonate during an MRI scan whilst the neonate's navel is continuously accessible for visual inspection and medical treatment, said immobilizer comprising:
an upper torso segment comprising an upper torso dorsal side having an upper torso lower flank and an upper torso upper flank extending therefrom and configured such that when the upper torso upper flank is wrapped on top of the upper torso lower flank with the neonate positioned therein, an upper torso section of the neonate is completely covered by the upper torso segment;
a lower torso segment comprising a lower torso dorsal side having a lower torso lower flank and a lower torso upper flank extending therefrom and configured such that when the lower torso upper flank is wrapped on top of the lower torso lower flank with the neonate positioned therein, a lower torso section of the neonate is completely covered by the lower torso segment;
a median naval segment comprising:
a) a median naval dorsal side connected to the upper torso dorsal side and the lower torso dorsal side,
b) a median naval lower flank having a first u-shaped cut out positioned between and connected to the upper torso lower flank and the lower torso lower flank,
c) a median naval upper flank having a second u-shaped cut out connected to the upper torso upper flank and the lower torso upper flank,
wherein the median naval lower flank and the median naval upper flank are configured such that when the median naval upper flank is wrapped on top of the median naval lower flank with the neonate positioned therein, the first u-shaped cut out and the second u-shaped cut out form an access aperture, the access aperture allowing a median naval section of the neonate to visible and accessible; and
a head restraining segment comprising at least two noise reduction components, the noise reduction components are coupled to the head restraining segment such that when the head restraining segment is wrapped around the head of the neonate the at least two noise reduction components are positioned over a respective ear of the neonate.

2. The neonate immobilizer of claim 1 wherein the at least two noise reduction components are cushions that can reduce the acoustic noise level by 2-8 db(A).

3. The neonate immobilizer of claim 1 wherein the at least two noise reduction components are positioned between an outer fabric layer of the head restraining segment and an inner fabric layer of the head restraining system.

4. The neonate immobilizer of claim 1 wherein the at least two noise reduction components are circular shaped, oval shaped, ear shaped, or any combination thereof.

5. The neonate immobilizer of claim 1 wherein the at least two noise reduction components are melamine

6. The neonate immobilizer of claim 1 wherein the at least two noise reduction components have a size based on a size of the neonate to be imaged.

7. The neonate's immobilizer according to claim 1, wherein said immobilizer is made of MRI-proof materials.
